SEQUENCE LISTING (5 sequences)

SEQ ID NO: 1 - protein, 5 aa, Artificial Sequence
  Synthetic
VPGXG

SEQ ID NO: 2 - protein, 5 aa, Artificial Sequence
  Synthetic
PGXGV

SEQ ID NO: 3 - protein, 5 aa, Artificial Sequence
  Synthetic
GXGVP

SEQ ID NO: 4 - protein, 5 aa, Artificial Sequence
  Synthetic
XGVPG

SEQ ID NO: 5 - protein, 5 aa, Artificial Sequence
  Synthetic
GVPGX

(19) United States
(12) Patent Application Publication
(10) Pub. No.: US 2015/0182627 A1
(43) Pub. Date: Jul. 2, 2015

(54) LIPOSOME INCLUDING ACTIVE INGREDIENT AND IMAGING AGENT AND USE THEREOF

(71) Applicant: SAMSUNG ELECTRONICS CO., LTD., SUWON-SI (KR)

(72) Inventors: Hyun-ryoung KIM, Guri-si (KR); Sang-joon PARK, Seongnam-si (KR); Eun-sung PARK, Seongnam-si (KR)

(21) Appl. No.: 14/587,746

(22) Filed: Dec. 31, 2014

(30) Foreign Application Priority Data
Dec. 31, 2013 (KR) 10-2013-0168834

(51) Int. Cl. A61K 47/24; A61K 9/127; A61K 49/10; A61K 31/704; A61K 49/18

(52) U.S. Cl. CPC A61K 47/24; A61K 31/704; A61K 49/1812; A61K 49/10; A61K 9/127

(57) ABSTRACT

A stimulus-sensitive liposome with a lipid bilayer comprising a first imaging agent, and an active ingredient and second imaging agent in an interior space defined by the lipid bilayer; a composition including the liposome; and a method of monitoring delivery and release of the active ingredient to a target site of an individual by using the liposome.

### RELATED APPLICATIONS

This application claims the benefit of Korean Patent Application No. 10-2013-0168834, filed on Dec. 31, 2013, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 1,505 bytes ASCII (Text) file named “718135_ST25.TXT,” created Dec. 22, 2014.

### BACKGROUND

1. Field

The present disclosure relates to a liposome including an active ingredient and an imaging agent, a composition including the liposome, and a method of monitoring delivery and release of the active ingredient to a target site of an individual by using the composition.

2. Description of the Related Art

In cancer treatment, the effect of a drug or a treatment prognosis may vary depending on properties of the blood vessels of a cancer patient. In a case of a patient in which many blood vessels are distributed around a cancer cell (e.g., a patient with cancerous tumor cells that have induced angiogenesis), a drug may easily access the cancer cell and thereby the treatment effect may be high. On the other hand, when there are not many blood vessels around a cancer cell, effect of a drug may be much lower.

Mild hyperthermia is a treatment method in which temperature at a cancer region is maintained from about 42 to about 45° C. to induce damage to the cancer. Mild hyperthermia may be paired with a therapy that involves the administration of a drug-loaded carrier that bursts when heat is applied. As a result, the drug loaded carrier releases the loaded drug only at a specific mild hyperthermia site.

Drug tracking refers to measuring how much drug is delivered and whether a drug has been accurately administered to a target site (e.g., a cancerous tumor). Until now, information about how much drug is accumulated at a cancer site in each patient has not been obtainable.

Therefore, there is still need for method of verifying whether a drug is delivered to a desired site and whether a desired amount of a drug is released at the desired site.

### SUMMARY

Provided is a stimulus-sensitive liposome comprising a lipid bilayer comprising a first imaging agent, and defining an interior space of the stimulus-sensitive liposome; and an active ingredient and a second imaging agent contained in the interior space, wherein the first imaging agent is a T1 imaging agent and the second imaging agent is a T2 imaging agent, or the first imaging agent is a T2 imaging agent and the second imaging agent is a T1 imaging agent; as well as a composition comprising the liposome and a carrier.

Also provided is a method of monitoring delivery and release of an active ingredient to a target site of an individual. The method comprises administering to an individual a stimulus-sensitive liposome, wherein the liposome comprises a lipid bilayer comprising of the stimulus-sensitive liposome includes a first imaging agent, and defining an inner interior space of the stimulus-sensitive liposome; and includes an active ingredient and a second imaging agent contained in the interior space, wherein the first imaging agent is a T1 imaging agent and the second imaging agent is a T2 imaging agent, or the first imaging agent is a T2 imaging agent and the second imaging agent is a T1 imaging agent; imaging the first imaging agent at the target site of an the individual to monitor the delivery of the liposome to the target site; heating the liposome at the target site to release the active ingredient and the second imaging agent; and imaging the second imaging agent at the target site to monitor the release of the active ingredient to the target site.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram showing a method of monitoring drug delivery and release using the liposome prepared according to an embodiment of the present invention;

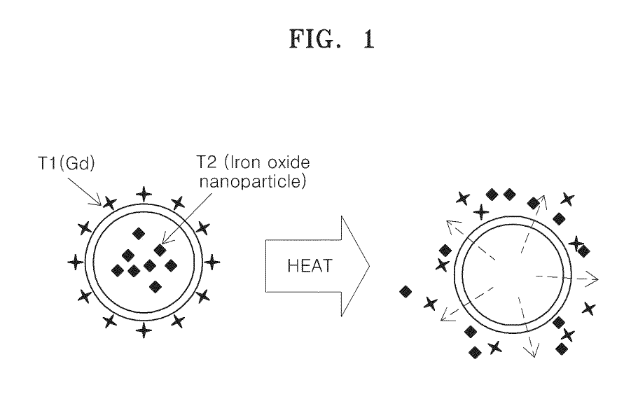

FIG. 2A provides a TEM image of iron oxide nanoparticles enveloped in liposomes prepared according to an embodiment of the present invention (Gd-STL-002-IO (5 nm)) and a graph of the diameter of the liposomes as determined by dynamic light scattering;

FIG. 2B provides a TEM image of iron oxide nanoparticles enveloped in liposomes prepared according to an embodiment of the present invention (Gd-STL-002-IO (10 nm)) and a graph of the diameter of the liposomes as determined by dynamic light scattering;

FIG. 3A is a graph of doxorubicin release from the Gd-STL-002-IO (5 nm) liposome prepared according to an embodiment of the present invention at various temperatures.

FIG. 3B is a graph of drug release of a conventional doxorubicin-containing liposome (lysolipid thermally sensitive liposome: LTSL) at various temperatures;

FIG. 4A is a graph of doxorubicin release over time from the Gd-STL-002-IO (5 nm) liposome prepared according to an embodiment of the present invention in a 20% blood serum at 37° C.;

FIG. 4B is a graph of drug release over time from a conventional doxorubicin-containing liposome (LTSL);

FIG. 5A provides a T1 weighted MR image at 37° C. and a T2 weighted MR image at 42° C. obtained from a liposome prepared according to an embodiment of the present invention;

FIG. 5B is a graph comparing the ROI (region of interest) values of the T1 weighted MR image and the T2 weighted MR image of FIG. 5A.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An aspect of the present invention provides a stimulus-sensitive liposome comprising a lipid bilayer. The lipid bilayer includes a first imaging agent (i.e., the first imaging agent is part of the lipid bilayer itself). The lipid bilayer also defines an interior space of the stimulus-sensitive liposome, and the liposome includes an active ingredient and a second imaging agent contained within the interior space. In the liposome, the first imaging agent may be a T1 imaging agent and the second imaging agent may be a T2 imaging agent. Or, the first imaging agent may be a T2 imaging agent and the second imaging agent may be a T1 imaging agent. The first imaging agent may be conjugated with a lipid forming the lipid bilayer of the liposome. The first imaging agent may be exposed to the outside of the liposome (e.g., located partially or completely on an outer surface of the liposome).

The term “liposome” as used herein refers to an artificially prepared vesicle including a lipid bilayer. A liposome may be a unilamellar vesicle (i.e., a liposome bounded by a single lipid bilayer) or a multilamellar vesicle.

The term “lipid bilayer” used herein refers to a membrane composed of two layers of lipid molecules. A lipid bilayer may have a thickness similar to that of a naturally existing membrane, for example, a cell membrane, a nuclear membrane, or a viral envelope. For example, the thickness of the lipid bilayer may be about 10 nm or less, for example, from about 1 nm to about 9 nm, from about 2 nm to about 8 nm, from about 2 nm to about 6 nm, from about 2 nm to about 4 nm, or from about 2.5 nm to about 3.5 nm. A lipid bilayer is a barrier that contains ions and larger molecules (e.g., proteins) and prevents them from diffusing. The “lipid molecule” included in the lipid bilayer may be a molecule having a hydrophilic head and a hydrophobic tail (e.g., phospholipid). The lipid molecule may be a molecule comprising carbon atoms of about C12 to about C50. The carbon atoms may be distributed in one or more carbon chains.

The term “imaging agent” used herein refers to a substance which is used to artificially increase the difference of energy (e.g., X-ray) absorption between tissues to increase imaging contrast so that tissues or blood vessels may be viewed clearly during magnetic resonance (MR) imaging or computed tomography. MR imaging agents are classified as a positive contrast (i.e., T1) imaging agent and as a negative contrast (i.e.,T2) imaging agent.

A T1 imaging agent refers to a substance which reduces T1 relaxation time to increase signal strength in a T1 weighted image. A T1 imaging agent includes a paramagnetic metal ion.

A T2 imaging agent refers to a substance which reduces T2 relaxation time to increase signal strength in a T2 weighted image. A T2 imaging agent may reduce both the T1 relaxation time and the T2 relaxation time. A T2 imaging agent includes a nanoparticle. The nanoparticle may include a superparamagnetic (SPM) substance.

The term “active ingredient” used herein refers to a biologically active substance. The active ingredient may be a compound, a protein, a peptide, a nucleic acid, a nanoparticle, or a combination thereof. The active ingredient may comprise an anticancer agent, an anti-angiogenesis agent, an anti-inflammatory agent, an analgesic, an antiarthritic, a sedative, an antidepressant, an antipsychotic drug, a tranquilizer, an anxiolytic, a narcotic antagonist, an antiparkinsonian, a cholinergic agonist, an immunosuppressant, an antiviral agent, an antibiotics, an anorectic agent, an anticholinergic agent, an antihistamine, an anti-migraine agent, a hormone agent, a vasodilator, a contraceptive, an antithrombotic, a diuretic, an antihypertensive agent, a cardiovascular disease therapeutic agent, an anti-wrinkle agent, a skin anti-aging agent, a skin-whitening agent, or a combination thereof.

The stimulus-sensitive liposome may be a liposome in which release of a substance loaded therein may be controlled (i.e., caused by) by a stimulus. The stimulus-sensitive liposome may be, for example, a temperature-sensitive liposome, a pH-sensitive liposome, a chemosensitive liposome, a radiation-sensitive liposome, an ultrasound-sensitive liposome, or a combination thereof. The temperature-sensitive liposome, pH-sensitive liposome, chemosensitive liposome, radiation-sensitive liposome, and ultrasound-sensitive liposome may release a substance loaded therein in an environment where there is a specific temperature, a specific pH, a chemical, radiation, or ultrasonic irradiation, respectively, that is applied to the liposome.

The lipid bilayer may include a phospholipid, a phospholipid derivative derivatised with a hydrophilic polymer, a stabilizer, an elastin-like polypeptide, or a combination thereof.

The term “phospholipid” used herein refers to a complex lipid including a phosphate-ester. A phospholipid is a main component of a biological membrane such as a cell membrane, an endoplasmic reticulum, a mitochondrion, and a myelin sheath surrounding a nerve fiber. A phospholipid has a hydrophilic head and two hydrophobic tails.

The phospholipid may be phosphatidyl choline, phosphatidyl glycerol, phosphatidylinositol, phosphatidylenthanolamine, or a combination thereof. Phosphatidyl choline includes choline as a head group and glycerophosphoric acid as a tail. Glycerophosphoric acid may be a saturated or an unsaturated fatty acid. Glycerophosphoric acid may have carbon atoms of from C14 to C50. The phosphatidyl choline may be 1,2-dipalmitoyl-sn-glycero-3-phosphocholine (DPPC), 1,2-distearoyl-sn-glycero-3-phosphocholine (DSPC), egg phosphatidyl choline, soy phosphatidyl choline, or a combination thereof. The phospholipid may have a DPPC to DSPC ratio of, for example, from about 5:1 to about 1:5, from about 4:1 to about 1:4, from about 3:1 to about 1:3, or from about 2:1 to about 1:2.

The hydrophilic polymer may be polyethylene glycol, polylactic acid, polyglycol acid, a polylactic acid-polyglycol acid copolymer, polyvinyl alcohol, polyvinyl pyrrolidone, oligosaccharide, or a combination thereof. A phospholipid derivative derivatised with a hydrophilic polymer may be, for example, 1,2-distearoylphosphatidylethanolamine-methyl-polyethylene glycol (DSPE-PEG). The lipid bilayer may include both a phospholipid and a phospholipid derivative. In the lipid bilayer, the ratio of the phospholipid to the phospholipid derivative may be, for example, from about 55:1 to about 55:3, from about 55:1.5 to about 55:2.5, or 55:1.8 to about 55:2.2, for example, 55:2.

The stabilizer may be a sterol or a sterol derivative, a sphingolipid or a sphingolipid derivative, or a combination thereof. The stabilizer may be cholesterol, β-cholesterol, sistosterol, erogsterol, stigmasterol, 4,22-stigmastadien-3-on, stigmasterol acetate, lanosterol, or a combination thereof. The stabilizer may be, for example, cholesterol. The stabilizer may strengthen a lipid bilayer and help to decrease permeability of the lipid bilayer. The lipid bilayer may include both the phospholipid and the stabilizer. In the lipid bilayer, the ratio of the lipid bilayer to the stabilizer, for example, cholesterol may be from about 14:1 to about 5:1, for example, 11:2.

The term “elastin-like polypeptide” (ELP) used herein refers to a type of amino acid polymer of which conformation is changed by temperature. The ELP may be a polymer having inverse phase transitioning behavior. The term “inverse phase transitioning behavior” refers to becoming soluble in an aqueous solution when the temperature is lower than an inverse phase transition temperature (Tt) and becoming insoluble in an aqueous solution when the temperature is higher than the Tt. As the temperature of an ELP increases, the ELP may change the conformation thereof from a highly soluble elongated chain to a tightly folded aggregate having a much lower solubility. Such an inverse phase transition behavior may be induced as the ELP structure includes a greater portion of a β-turn structure and a distorted β-structure due to a temperature increase. When an ELP is bound to a composition of a lipid bilayer, the lipid bilayer may be disrupted and destroyed as the temperature is increased from a temperature lower than the Tt of the ELP to a temperature higher than the Tt of the ELP.

The term “phase transition temperature” used herein refers to a temperature at which a phase of a substance is changed from a solid phase to a liquid phase or from a liquid phase to a solid phase. Destruction of a lipid bilayer may be dependent on a phase transition temperature of the lipid bilayer itself. A temperature at which an active ingredient included in a liposome is released may be controlled by controlling a lipid phase transition temperature and/or an ELP inverse phase transition temperature. The lipid bilayer may include a phospholipid, a phospholipid derivative, a stabilizer, and/or an ELP. A phase transition temperature of a lipid bilayer or a liposome including the ELP may be, for example, from about 25° C. to about 70° C., from about 25° C. to about 65° C., from about 25° C. to about 60° C., from about 25° C. to about 55° C., from about 25° C. to about 50° C., from about 30° C. to about 50° C., from about 35° C. to about 50° C., from about 37° C. to about 50° C., from about 37.5° C. to about 50° C., from about 38° C. to about 45° C., from about 38.5° C. to about 45° C., or from about 39° C. to about 45° C.

The ELP may be include at least one repeated unit selected from the group consisting of VPGXG (SEQ ID NO: 1), PGXGV (SEQ ID NO: 2), GXGVP (SEQ ID NO: 3), XGVPG (SEQ ID NO: 4), GVPGX (SEQ ID NO: 5), and a combination thereof, and wherein V is valine, P is proline, G is glycine, and X is any amino acid except proline. Each X in a repeated unit may be the same amino acid or a different amino acid. The selected repeated unit may be repeated at least two times, for example, from about two times to about 200 times.

The T1 imaging agent may be a metal, a metal compound, a metal complex, and a combination thereof. The metal compound may be ionic compound or non-ionic compound. The metal complex may be a coordination complex. The metal may be a transition metal. The transition metal may be La, Pr, Nd, Gd, Tb, Mn, Zn, Fe, Sc, Ti, V, Zn, Y, Zr, Nb, Mo, Pd, Ag, Au, Cd, W, or Re. The transition metal may be, for example, Gd, iron oxide, Mn, or Au. The T2 imaging agent may be a metal compound or a metal nanoparticle. The diameter of the metal nanoparticle may be from about 1 nm to about 10 nm, from about 2 nm to about 9 nm, from about 3 nm to about 8 nm, from about 3.5 nm to about 7 nm, from about 3.5 nm to about 6.5 nm, from about 4.0 nm to about 6.0 nm, from about 4.2 nm to about 5.8 nm, from about 4.5 nm to about 5.5 nm, or from about 4.8 nm to about 5.2 nm.

The T1 imaging agent may be a gadolinium ion (Gd³⁺) or a gadolinium complex. The gadolinium complex may be, for example, gadoteric acid, gadodiamide, gadobenic acid, gadopentetetic acid, gadoteridol, gadoversetamide, gadoxetatic acid, gadobutrol, or a combination thereof. The gadolinium complex may be, for example, 1,2-dioctadecanoyl-sn-glycero-3-phosphoethanolamine-N-diethylenetriaminepentaacetic acid (gadolinium salt) (DSPE-DTPA (Gd)), 1,2-dihexadecanoyl-sn-glycero-3-phosphoethanolamine-N-diethylenetriaminepentaacetic acid (gadolinium salt) (DPPE-DTPA (Gd)), or 1,2-ditetradecanoyl-sn-glycero-3-phosphoethanolamine-N-diethylenetriaminepentaacetic acid (gadolinium salt) (DMPE-DTPA (Gd)), or a combination thereof.

The T2 imaging agent may be an iron oxide nanoparticle.

The active ingredient may be methotrexate, doxorubicin, epirubicin, daunorubicin, vincristine, vinblastine, etoposide, ellipticine, camptothecin, doxetaxel, paclitaxel, cisplatin, prednisone, methyl-prednisone, biprofen, idarubicin, valrubicin, mitoxantrone, ampicillin, streptomycin, penicillin, or a combination thereof.

In the lipid bilayer of the liposome, the ratio of a phospholipid to a first imaging agent may be from about 95:6 to about 95:1, from about 95:5.5 to about 95:1, from about 95:5.3 to about 95:1, from about 95:5.1 to about 95:1, or from about 95:5 to about 95:1.

The diameter of the liposome may be, for example from about 50 nm to about 500 nm, from about 50 nm to about 400 nm, from about 50 nm to about 300 nm, from about 50 nm to about 200 nm, or from about 50 nm to about 150 nm.

Another aspect of the present invention provides a composition including a stimulus-sensitive liposome as described herein. The composition may be used to deliver an active ingredient to a target site of an individual. The first imaging agent, the lipid bilayer, the active ingredient, the second imaging agent, the stimulus-sensitive liposome, the T1 imaging agent, and the T2 imaging agent are as described, above.

The composition may further include a pharmaceutically acceptable carrier or diluent. The pharmaceutically acceptable carrier or diluent may be known in the art. The carrier or diluent may be lactose, dextrose, sucrose, sorbitol, mannitol, starch, acacia rubber, calcium phosphate, alginate, gelatin, calcium silicate, microcrystalline cellulose, polyvinylpyrrolidone, cellulose, water (e.g., saline solution and sterilized water), syrup, methyl cellulose, methyl hydroxybenzoate, propylhydroxybenzoate, talc, magnesium stearate, mineral oil, Ringer's solution, buffer, maltodextrin solution, glycerol, ethanol, or a combination thereof. The composition may further include a lubricant, a wetting agent, a sweetening agent, a flavoring agent, an emulsifier, a suspending agent, or a preservative.

The composition may be formulated according to a method known in the art in a unit dosage or in a multi-dose container by using a pharmaceutically acceptable carrier and/or excipient. The dosage form may be a solution, a suspension, syrup, or an emulsion in an oily or aqueous medium or an extract, powders, a powdered drug, a granule, a tablet, or a capsule. The dosage form may further include a dispersing agent or a stabilizer. The aqueous medium may include a saline solution or a phosphate buffered saline.

Another aspect of the present invention provides a method of monitoring delivery and release of an active ingredient to a target site of an individual including administering to a target site of an individual a temperature-sensitive liposome wherein the liposome comprises a lipid bilayer comprising a first imaging agent, and defining an interior space of the temperature-sensitive liposome; and an active ingredient and a second imaging agent contained in the interior space, wherein the first imaging agent is a T1 imaging agent and the second imaging agent is a T2 imaging agent, or the first imaging agent is a T2 imaging agent and the second imaging agent is a T1 imaging agent; imaging the first imaging agent at the target site of an individual to monitor the delivery of the liposome to the target site; heating the liposome at the target site to release the active ingredient and the second imaging agent; and imaging the second imaging agent at the target site to monitor the release of the active ingredient to the target site.

The first imaging agent, the lipid bilayer, the active ingredient, the second imaging agent, and the stimulus-sensitive liposome are described above. In the liposome, the first imaging agent may be a T1 imaging agent and the second imaging agent may be a T2 imaging agent. Or, the first imaging agent may be a T2 imaging agent and the second imaging agent may be a T1 imaging agent. The first imaging agent may be conjugated with a lipid constituting the lipid bilayer. The first imaging agent may be exposed to an outside of the liposome. The T1 imaging agent and the T2 imaging agent are described above.

The individual may a mammal including a human.

The administering may be oral administration or parenteral administration. The parenteral administration may be, for example, intravenous injection, hypodermic injection, intramuscular injection, intracoelomic (abdominal cavity, joint, or optical) injection, or direct injection. The direct injection may be a direct injection to a disease symptom site, for example, a tumor site. The liposome may be injected to blood such as venous blood and delivered by a flow of blood to a target site such as a tumor site. The target site may be leaky. The amount of administration may be variously prescribed depending on such factors as formulation method, administration method, age, weight, sex, morbid condition, and food intake of a patient, administration time, administration pathway, excretion rate, and response sensitivity. The amount of administration may be, for example, from about 0.001 mg/kg to about 100 mg/kg

The method may include imaging a first imaging agent at the target site (e.g., imaging the target site with a method that detects the first imaging agent). When the first imaging agent is a T1 imaging agent, the imaging may involve obtaining a T1 weighted image of the target site (e.g., imaging by a method that detects the T1 imaging agent). When the first imaging agent is a T2 imaging agent, the imaging may involve obtaining a T2 weighted image of the target site (e.g., imaging by a method that detects the T2 imaging agent). Through the imaging with respect to the first imaging agent, accumulation of the liposome into the target site may be monitored. Signal magnitude at a region of interest (ROI) of the target site may be measured to monitor whether the liposome is delivered to or accumulated at the target site and to monitor the degree of the delivery or accumulation of the liposome.

The method includes heating the target site to release from the liposome the active ingredient and the second imaging agent. The heating may be performed after verifying accumulation of a desired amount of the liposome at the target site through imaging of the first imaging agent at the target site.

The heating may be heating to a temperature from about 39° C. to about 45° C. The heating may be performed, for example, by application of high intensity focused ultrasound (HIFU). Before the heating, the lipid bilayer of the liposome may be maintained without destruction. Therefore, before the heating, the active ingredient and the second imaging agent are not released from the liposome. Through the heating, the active ingredient and the second imaging agent may be simultaneously released from the liposome.

The method may include imaging the second imaging agent at the target site (e.g., imaging the target site with a method that detects the first imaging agent). When the second imaging agent is a T1 imaging agent, the imaging may involve obtaining a T1 weighted image at the target site (e.g., imaging the target set with a method that detects a T1 imaging agent). When the second imaging agent is a T2 imaging agent, the imaging may involve obtaining a T2 weighted image at the target site (e.g., imaging the target set with a method that detects a T2 imaging agent). Through the imaging with respect to the second imaging agent, release of the active ingredient to the target site may be monitored. Signal magnitude at a region of interest (ROI) of the target site may be measured to monitor whether the second imaging agent is released and the degree of the release, through which whether the active ingredient released together with the second imaging agent is released to the target site and the degree of the release may be verified.

Hereinafter, the present invention will be described in further detail with reference to examples. It will be obvious that these examples are illustrative purposes only and are not to be construed to limit the scope of the present invention.

### EXAMPLE 1

### Preparation of Liposome

1,2-dipalmitoyl-sn-glycero-3-phosphocholine (DPPC), 1,2-distearoyl-sn-glycero-3-phosphocholine (DSPC), 1,2-dioctadecanoyl-sn-glycero-3-phosphoethanolamine-N-diethylenetriaminepentaacetic acid (gadolinium salt) (DSPE-DTPA (Gd)), 1,2-distearoyl-sn-glycero-3-phosphoethanolamine-N-[methoxy(polyethylene glycol)-2000] (ammonium salt) (DSPE-PEG), cholesterol, and stearoyl-VPGVG VPGVG VPGVG-NH2 (hereinafter referred to also as “SA-V3-NH2”) were used in the molar ratio of 41.25:11:2.75:2:10:0.55 to prepare a liposome having the shape of a unilamellar vesicle.

### (1.1) Preparation of Liposome by Room Temperature Preparation Method

In room temperature preparation method, SA-V3-NH2 (Peptron, Inc.) was dissolved in ethanol, and DPPC (Avanti Polar lipids, Inc.), DSPC (Avanti Polar lipids, Inc.), DSPE-PEG (Avanti Polar lipids, Inc.), and cholesterol (Avanti Polar lipids, Inc.) were dissolved in chloroform. DSPE-DTPA (Gd) (Avanti Polar lipids, Inc.) was dissolved in a mixed solution of cholesterol and ethanol. The ethanol solution and the chloroform solution were mixed in a round-bottom flask and then the solvent was evaporated at room temperature by using a rotary evaporator to form a lipid film on the inner wall of the round-bottom flask.

To the round-bottom flask, 250 mM ammonium sulfate solvent (pH 4.0) in which 1 mg/ml iron oxide (Fe₃O₄) nanoparticle, which is a T2 imaging agent, is dissolved was added to hydrate the lipid film. The hydrated solution was treated by vortexing and sonication.

The hydrated solution was sequentially extruded at room temperature by using Avanti® Mini-Extruder (Avanti Polar Lipids, Inc.) including polycarbonate membranes having a pore size of 400, 200, or 100 nm to prepare a liposome having the shape of a unilamellar vesicle. The prepared liposome solution was passed through a PD-10 (GE Healthcare) desalting column while providing 25 mM Tris HCI solution (pH 9.0) in the column to remove the iron oxide (Fe₃O₄) nanoparticles which were not enveloped.

According to the ammonium sulfate gradient method, doxorubicin was loaded in the inside of the liposome. In the state where the inside of the liposome is filled with an ammonium sulfate solvent (250 mM, pH 4.0) and the outside of the liposome is filled with a Tris-HCl buffer (25 mM, pH 9.0), 0.5 mg/ml of doxorubicin was added to the liposome solution and the mixed solution was incubated in a Thermomixer comfort (Eppendorf AG) at 37° C. for one hour.

The prepared liposome solution was passed through a PD-10 (GE Healthcare) desalting column while providing a phosphate buffered saline solution to the column to remove doxorubicin which was unloaded. As a result, prepared was a liposome wherein DSPE-DTPA (Gd) was attached to the lipid bilayer of the liposome, and doxorubicin and iron oxide (Fe₃O₄) nanoparticles were loaded in the inside of the liposome.

### (1.2) Preparation of Liposome by High Temperature Preparation Method

DPPC (Avanti Polar lipids, Inc.), DSPC (Avanti Polar lipids, Inc.), DSPE-PEG (Avanti Polar lipids, Inc.), and cholesterol (Avanti Polar lipids, Inc.) were dissolved in chloroform. DSPE-DTPA (Gd) (Avanti polar lipid, Inc.) was dissolved in a mixture of cholesterol and ethanol. The ethanol and the chloroform were mixed in a round-bottom flask and then the solvent was evaporated at room temperature by using a rotary evaporator to form a lipid film on the inner wall of the round-bottom flask.

To the round-bottom flask, 250 mM ammonium sulfate solvent (pH 4.0) in which 1 mg/ml iron oxide (Fe₃O₄) nanoparticle, which is a T2 imaging agent, is dissolved was added to hydrate the lipid film. The hydrated suspension underwent vortexing and then was treated with a sonicator of which temperature was set to be 60° C.

The hydrated suspension was sequentially extruded at 60 by using Avanti® Mini-Extruder (Avanti Polar Lipids, Inc.) including polycarbonate membranes having a pore size of 400, 200, or 100 nm to prepare a liposome having the shape a unilamellar vesicle. The prepared liposome was passed through a PD-10 (GE Healthcare) desalting column while providing 25 mM Tris HCl solution (pH 9.0) in the column to remove the iron oxide (Fe₃O₄) nanoparticles which were enveloped.

According to the ammonium sulfate gradient method, doxorubicin was loaded in the inside of the liposome. After PD-10 column, there are the inside of the liposome is filled with an ammonium sulfate solvent (250 mM, pH 4.0) and the outside of the liposome is filled with a Tris-HCI buffer (25 mM, pH 9.0), 0.5 mg/ml of doxorubicin was added to the liposome and the mixed suspension was incubated in a thermomixer comfort at 37° C. for one hour.

SA-V3-NH2 (Peptron, Inc.) was introduced to the prepared liposome solution by insertion. The SA-V3-NH2 was dissolved in water and the resulting solution was added to the liposome solution at a molar ratio of 1.1 with reference to the lipid. The prepared liposome was incubated in a thermomixer comfort (Eppendorf AG) at 25° C. for one hour.

The prepared liposome was passed through a PD-10 (GE Healthcare) desalting column while providing a phosphate buffered saline solution to the column to remove doxorubicin which was unloaded. As a result, prepared was a liposome wherein DSPE-DTPA (Gd) was attached to the lipid bilayer of the liposome and doxorubicin and iron oxide (Fe₃O₄) nanoparticles were loaded in the inside of the liposome.

### EXAMPLE 2

### Evaluation Physicochemical Properties of Liposome Prepared in Example 1

The diameter of the liposome obtained by varying the diameter of the iron oxide nanoparticles was measured to select an appropriate iron oxide nanoparticle.

According to the method of Example 1, a liposome including 5 nm of iron oxide (Fe₃O₄) nanoparticles (Gd-STL-002-IO (5 nm)) and a liposome including 10 nm of iron oxide (Fe₃O₄) nanoparticles (Gd-STL-002-IO (10 nm)) were respectively prepared. Then, a dynamic light scattering (DLS) analyzer (Malvern Instruments Ltd.) was used to measure the diameter of the liposomes.

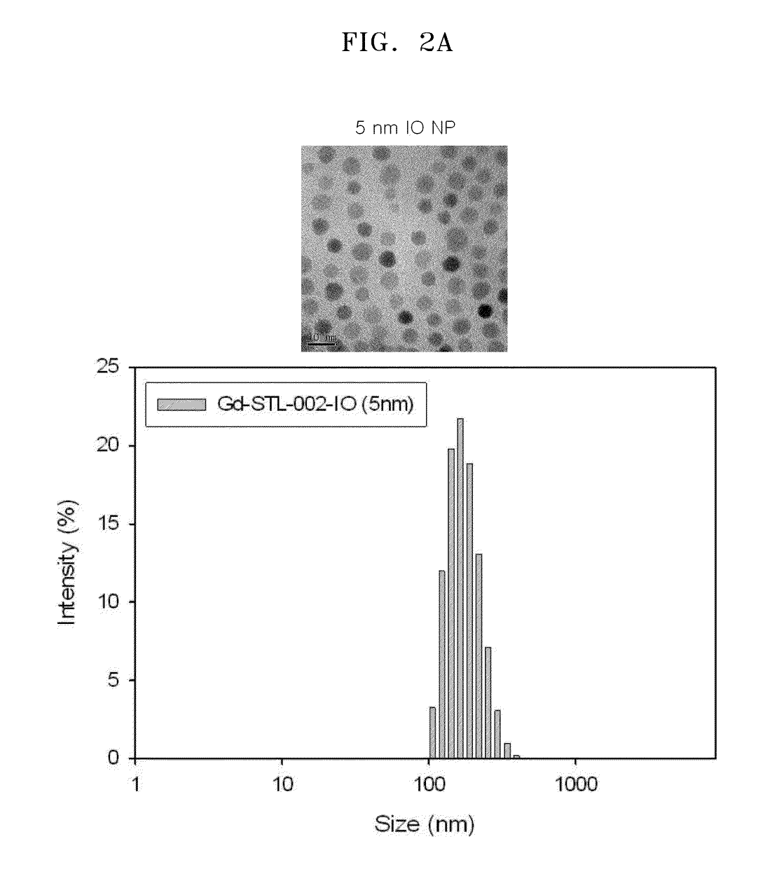

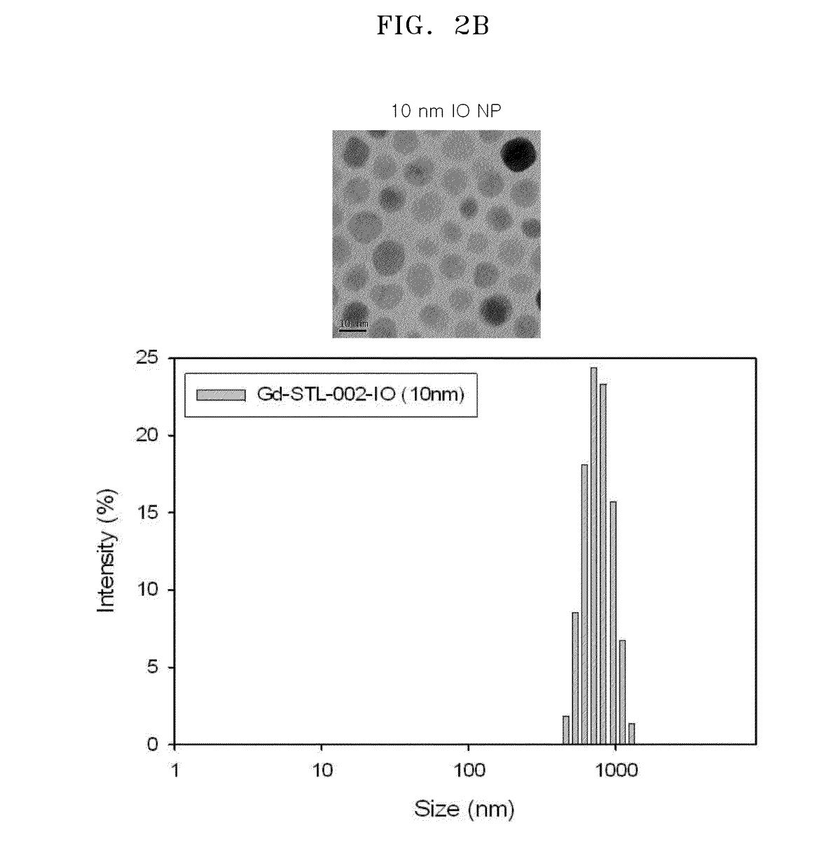

FIG. 2a shows the diameter of Gd-STL-002-IO (5 nm) liposome measured by dynamic light scattering. FIG. 2b shows the diameter of Gd-STL-002-IO (10 nm) liposome measured by dynamic light scattering.

In addition, Table 1 shows the physicochemical properties of the Gd-STL-002-IO (5 nm) liposome and the Gd-STL-002-IO (10 nm) liposome, respectively.

TABLE 1

| | | | Gd-STL-002-IO | |
|---|---|---|---|---|
| Property | Gd-STL-002-IO (5 nm) | | (10 nm) | |
| Lipid concentration | 10 | mg/ml | 10 | mg/ml |
| Liposome Diameter | 203 | nm | 776 | nm |
| Doxorubicin Load | 272 | μg/ml | 310 | μg/ml |
| Enveloped Iron | 1.3 | mM | 2.4 | mM |
| Concentration | | | | |
| Enveloped gadolinium | 0.22 | mM | 0.25 | mM |
| Concentration | | | | |

Considering the liposome diameter, the follow-up experiments were performed with the Gd-STL-002-IO (5 nm) liposome.

### EXAMPLE 3

### Measurement of Drug Release Profile and Evaluation of Liposome Stability at Different Liposome Temperatures

With the Gd-STL-002-IO (5 nm) liposome prepared in Example 1, the drug release profile and the liposome stability were measured according the temperature

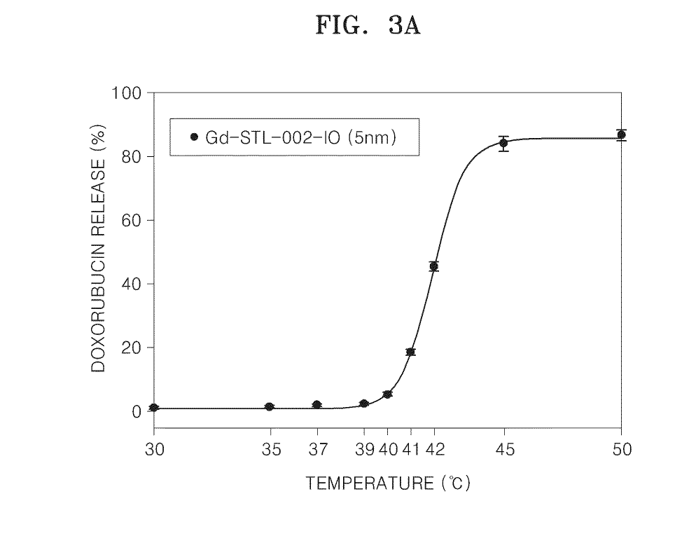

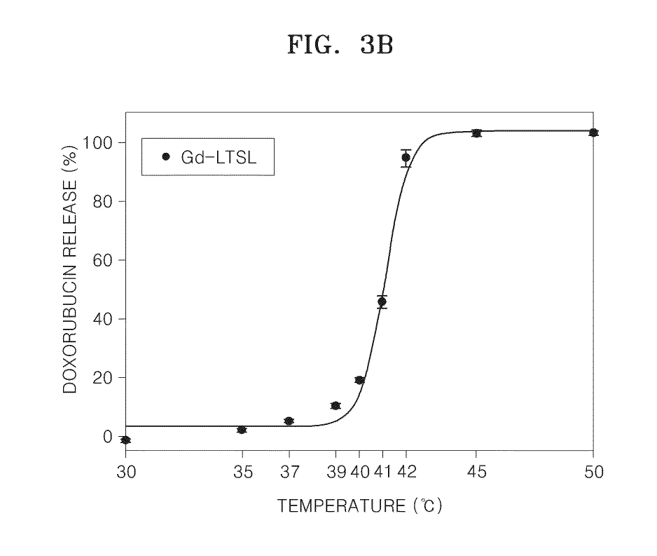

FIG. 3a shows the doxorubicin release profile of the Gd-STL-002-IO (5 nm) liposome prepared in Example 1 according the temperature FIG. 3b shows the drug release profile of a conventional doxorubicin-containing liposome (lysolipid thermally sensitive liposome: LTSL).

While the drug release began at about 37.8° C. from the conventional doxorubicin-containing liposome, the Gd-STL-002-IO (5 nm) liposome was very stable at 37° C. and thus there was almost no drug leakage. About 50% to 80% of the drug was released from the Gd-STL-002-IO (5 nm) liposome by a temperature stimulus of from about 42° C. to about 45° C.

The result verified that, in comparison with the conventional doxorubicin-containing liposome, the Gd-STL-002-IO(5 nm) liposome does not have a left shift of the Tt and thus the drug release from the Gd-STL-002-IO (5 nm) liposome may be controlled more stably and efficiently.

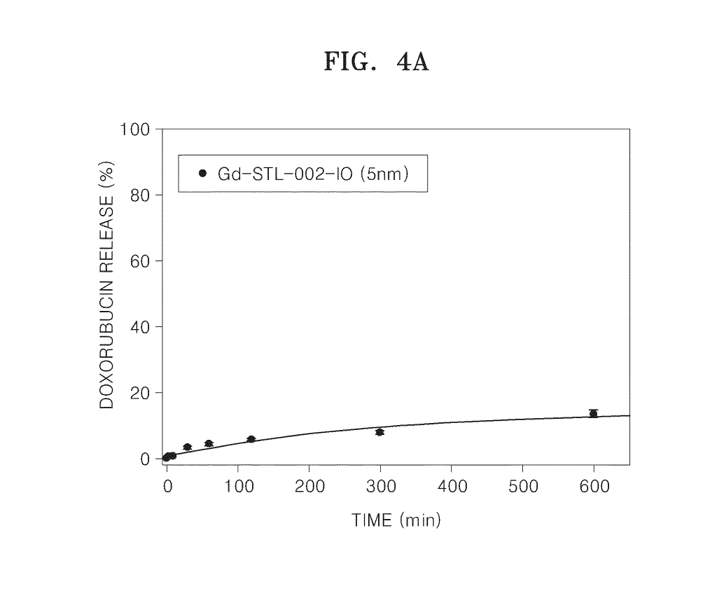

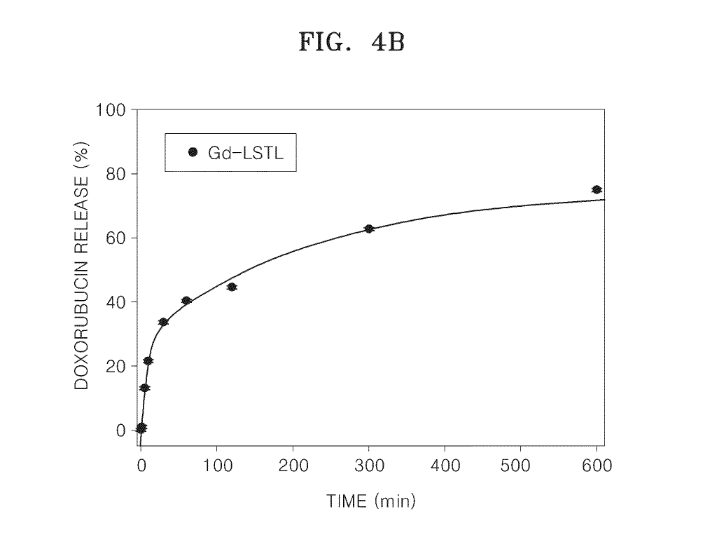

FIG. 4a shows the profile of doxorubicin release over time from the Gd-STL-002-IO (5 nm) liposome prepared according to Example 1 in a 20% blood serum at 37° C. As shown in FIG. 4a, the half-life of the liposome was longer than 10 hours. The result indicates that the liposome may be maintained as stable at 37° C. FIG. 4b shows the profile of doxorubicin release over time from the LTSL in a 20% blood serum at 37° C. The result indicates that LTLS is unstable showing 40% drug leakage after 1hr incubation.

### EXAMPLE 4

### Evaluation of MR Imaging Effect of Liposome

The imaging effect efficiency of the Gd-STL-002-IO(5 nm) liposome prepared in Example 1 was verified. T1 imaging was performed at 37° C. to evaluate the drug delivery monitoring function, and T2 imaging was performed at 42° C. to evaluate the drug release monitoring function.

The liposome was mixed with 1% Agarose gel solution at the ratio of 1:1, and the resulting suspension was incubated by using a thermomixer comfort (Eppendorf) at 37° C. and 42° C. for five minutes. Then, the temperature of the suspension was decreased to 25° C. to gelate the solution. Subsequently, a magnetic resonance imaging instrument (3.0 T Philips Intra Achieva, Philips) was used to verify the imaging effect of the liposome.

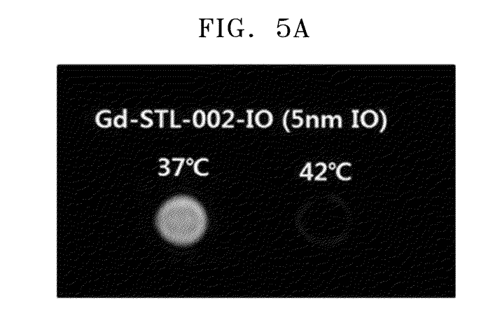

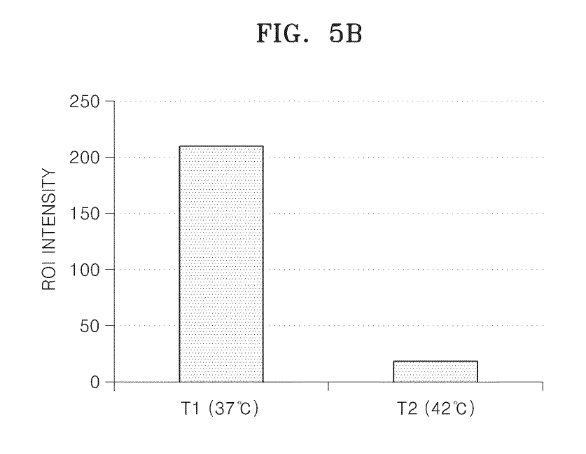

FIG. 5a shows the T1 weighted MR image at 37° C. and the T2 weighted MR image at 37° C. obtained from the Gd-STL-002-IO (5 nm) liposome of Example 1. FIG. 5b is a graph comparing the ROI values of the T1 weighted MR image and the T2 weighted MR image.

The Δ ROI value was 211 (37° C., T1) and 19 (42° C., T2), respectively, indicating that the ROI value of the T1 weighted MR image was about 11 times greater than that of the T2 weighted MR image. The result showed that both drug delivery monitoring through T1 imaging and drug release monitoring through T2 imaging are possible.

As described above, according to the one or more of the above embodiments of the present invention, a liposome including an active ingredient and an imaging agent, a composition including the same, and a method of monitoring delivery and release of the active ingredient to a target site of an individual by using the composition may be used to acquire drug delivery and release information in real-time, through which patient-customized medical service may be accomplished.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A stimulus-sensitive liposome comprising:
a lipid bilayer comprising a first imaging agent, and defining an interior space of the stimulus-sensitive liposome; and
an active ingredient and a second imaging agent contained in the interior space,
wherein the first imaging agent is a T1 imaging agent and the second imaging agent is a T2 imaging agent, or the first imaging agent is a T2 imaging agent and the second imaging agent is a T1 imaging agent.

2. The stimulus-sensitive liposome of claim 1, wherein the liposome is temperature-sensitive, pH-sensitive, chemosensitive, radiation-sensitive, ultrasound-sensitive, or a combination thereof.

3. The stimulus-sensitive liposome of claim 1, wherein the lipid bilayer comprises a phospholipid; a phospholipid comprising a hydrophilic polymer; a stabilizer; an elastin-like polypeptide; or a combination thereof.

4. The stimulus-sensitive liposome of claim 1, wherein the T1 imaging agent is a metal, a metal compound, a metal complex, or a combination thereof.

5. The stimulus-sensitive liposome of claim 1, wherein the T2 imaging agent is a metal nanoparticle.

6. The stimulus-sensitive liposome of claim 4, wherein the metal is gadolinium, iron oxide, manganese or gold.

7. The stimulus-sensitive liposome of claim 4, wherein the T1 imaging agent is a gadolinium complex compound.

8. The stimulus-sensitive liposome of claim 5, wherein the T2 imaging agent is an iron oxide nanoparticle.

9. The stimulus-sensitive liposome of claim 5, wherein the diameter of the nanoparticle is from about 1 nm to about 10 nm.

10. The stimulus-sensitive liposome of claim 1, wherein the active ingredient comprises methotrexate, doxorubicin, epirubicin, daunorubicin, vincristine, vinblastine, etoposide, ellipticine, camptothecin, doxetaxel, paclitaxel, cisplatin, prednisone, methyl-prednisone, biprofen, idarubicin, valrubicin, mitoxantrone, ampicillin, streptomycin, penicillin, or a combination thereof.

11. The stimulus-sensitive liposome of claim 3, wherein the ratio of the phospholipid to the first imaging agent is from about 95:5 to about 95:1.

12. The stimulus-sensitive liposome of claim 3, wherein the phospholipid comprises an elastin-like polypeptide, and the elastin-like polypeptide comprises at least one repeating unit selected from the group consisting of VPGXG (SEQ ID NO: 1), PGXGV (SEQ ID NO: 2), GXGVP (SEQ ID NO: 3), XGVPG (SEQ ID NO: 4), GVPGX (SEQ ID NO: 5), and a combination thereof, wherein X is any amino acid except proline.

13. The stimulus-sensitive liposome of claim 12, wherein the repeating unit is repeated from about two times to about 200 times.

14. The stimulus-sensitive liposome of claim 1, wherein the liposome has a phase transition temperature from about 39° C. to about 45° C.

15. The stimulus-sensitive liposome of claim 1, wherein the diameter of the liposome is from about 50 nm to about 500 nm.

16. A composition comprising the stimulus-sensitive liposome of claim 1 and a carrier.

17. A method of monitoring delivery and release of an active ingredient to a target site of an individual comprising:
administering to an individual a temperature-sensitive liposome wherein the liposome comprises a lipid bilayer comprising a first imaging agent, and defining an interior space of the temperature-sensitive liposome; and an active ingredient and a second imaging agent contained in the interior space, wherein the first imaging agent is a T1 imaging agent and the second imaging agent is a T2 imaging agent, or the first imaging agent is a T2 imaging agent and the second imaging agent is a T1 imaging agent;
imaging the first imaging agent at the target site of the individual to monitor the delivery of the liposome to the target site;
heating the liposome at the target site to release the active ingredient and the second imaging agent; and
imaging the second imaging agent at the target site to monitor the release of the active ingredient to the target site.

18. The method of claim 17, wherein the liposome is heated by heating the target site of the individual to a temperature of about 39° C. to about 45° C.

19. The method of claim 17, wherein the heating is performed by applying high intensity focused ultrasound (HIFU) to the target site.

20. The method of claim 17, wherein the active ingredient and the second imaging agent are simultaneously released from the liposome.
